(19) United States
(12) Patent Application Publication
(10) Pub. No.: US 2014/0005459 A1
(43) Pub. Date: Jan. 2, 2014

(54) HYDRODECHLORINATION OF IONIC LIQUID-DERIVED HYDROCARBON PRODUCTS

(71) Applicants: Bi-Zeng Zhan, Albany, CA (US); Hye Kyung Cho Timken, Albany, CA (US); Zunqing He, San Rafael, CA (US); Russell Evan Cooper, Martinez, CA (US)

(72) Inventors: Bi-Zeng Zhan, Albany, CA (US); Hye Kyung Cho Timken, Albany, CA (US); Zunqing He, San Rafael, CA (US); Russell Evan Cooper, Martinez, CA (US)

(73) Assignee: CHEVRON CORPORATION, San Ramon, CA (US)

(21) Appl. No.: 14/013,631

(22) Filed: Aug. 29, 2013

Related U.S. Application Data
(62) Division of application No. 12/847,313, filed on Jul. 30, 2010.

(51) Int. Cl. C07C 7/12

(52) U.S. Cl. CPC C07C 7/12
USPC 585/823

(57) ABSTRACT

We provide a hydrodechlorination and hydrogen chloride recovery process, comprising:
a) contacting at least one hydrocarbon product with a hydrodechlorination catalyst in the presence of hydrogen under hydrodechlorination conditions to provide:
   i) an off-gas comprising an HCl, and
   ii) a dechlorinated product;
b) separating the dechlorinated product from the off-gas;
c) contacting the off-gas with an adsorbent under HCl adsorbing conditions such that the HCl is adsorbed by the adsorbent; and
d) after step c), recovering the HCl from the adsorbent.

This application is a divisional of U.S. patent application Ser. No. 12/847,313, filed Jul. 30, 2010, in Group Art Unit 1771; and herein incorporated in its entirety.

### TECHNICAL FIELD

The present invention relates to hydrodechlorination of ionic liquid derived hydrocarbon products.

### BACKGROUND

The conversion by refining industries of light paraffins and light olefins to more valuable cuts has been accomplished by the alkylation of paraffins with olefins and by the polymerization of olefins. Such processes, which have been used since the 1940's, continue to be driven by the increasing demand for high quality and clean burning high-octane gasoline, distillate and lubricating base oil.

Conventional alkylation processes use vast quantities of H₂SO₄ or HF as catalyst. The quest for an alternative catalytic system to replace the conventional catalysts has been researched by various groups in both academic and industrial institutions. Unfortunately, thus far, no viable replacement to the conventional processes has been commercialized.

Recently there has been considerable interest in metal halide ionic liquid catalysts as alternatives to conventional catalysts. As an example, the ionic liquid catalyzed alkylation of isoparaffins with olefins is disclosed in U.S. Pat. No. 7,432,408 to Timken et al. U.S. Pat. No. 7,572,943 to Elomari et al. discloses the ionic liquid catalyzed oligomerization of olefins and the alkylation of the resulting oligomers(s) with isoparaffins to produce alkylated olefin oligomers. The presence of HCl as a co-catalyst with an ionic liquid provides an increased level of catalytic activity, for example, as disclosed by the '408 patent. Typically, anhydrous HCl or organic chloride may be combined with the ionic liquid feed to attain the desired level of catalytic activity and selectivity (see, e.g., U.S. Pat. Nos. 7,495,144 to Elomari, and 7,531,707 to Harris et al.). When organic chloride is used as the co-catalyst with the ionic liquid, HCl may be formed in situ in the reactor during the hydrocarbon conversion process.

Hydrocarbon product(s) of ionic liquid catalyzed hydrocarbon conversions, such as alkylate or distillate or base oil, typically contain substantial amounts of organic chloride components that are produced during the reaction. The removal of organic chloride components from such hydrocarbon product(s) may be desirable, e.g., to prevent the formation of unwanted by-products during combustion of liquid fuels (see, for example, U.S. Pat. No. 7,538,256 to Driver et al., the disclosure of which is incorporated by reference herein in its entirety).

There is a need for processes for the efficient dechlorination of hydrocarbon products derived from ionic liquid catalyzed hydrocarbon conversion reactions. There is a further need for the removal of HCl from hydrodechlorination off-gas.

### BRIEF DESCRIPTION OF THE DRAWINGS

FIG. 1A represents a scheme for a combined hydrocarbon conversion, hydrodechlorination, and hydrogen chloride recovery process, according to an embodiment of the present invention;

FIG. 1B represents a scheme for a combined hydrocarbon conversion, hydrodechlorination, and hydrogen chloride recovery process, according to another embodiment of the present invention;

FIG. 2 shows the boiling point distribution of a hydrodechlorinated alkylate product, as compared with a chlorinated alkylate feed, according to an embodiment of the present invention; and

FIG. 3 shows an HCl breakthrough curve by contacting an HCl-containing off-gas from the hydrodechlorination of an alkylate distillate with an adsorbent comprising zeolite 4A.

### SUMMARY

The present invention provides processes for the hydrodechlorination of hydrocarbon products derived from ionic liquid catalyzed hydrocarbon conversion reactions. The present invention also provides processes for the recovery of HCl obtained from hydrodechlorination off-gas. The present invention further provides an integrated hydrocarbon conversion, hydrodechlorination, and HCl recovery process, wherein HCl that is recovered from dechlorination processes may be used as a catalyst promoter for the ionic liquid catalyzed hydrocarbon conversion reactions.

According to one aspect of the present invention there is provided an integrated hydrocarbon conversion process comprising contacting at least one hydrocarbon reactant with an ionic liquid catalyst in a hydrocarbon conversion zone under hydrocarbon conversion conditions to provide at least one hydrocarbon product comprising at least one halogenated component; and contacting the at least one hydrocarbon product with a hydrodechlorination catalyst in the presence of hydrogen in a hydrodechlorination zone under hydrodechlorination conditions to provide: i) a dechlorinated product, and ii) an off-gas comprising HCl. A first chloride content of the at least one hydrocarbon product may be greater than 50 ppm, the chloride content of the dechlorinated product is lower than the feed, to be less than 50 ppm, and typically less than 10 ppm.

In an embodiment, the present invention also provides a hydrogen chloride recovery process comprising contacting at least one hydrocarbon product with a hydrodechlorination catalyst in the presence of hydrogen under hydrodechlorination conditions to provide: i) an off-gas comprising HCl, and ii) a dechlorinated product; separating the dechlorinated product from the off-gas; contacting the off-gas with an adsorbent under HCl adsorbing conditions such that the HCl is adsorbed by the adsorbent; and, after the prior step, recovering the HCl from the adsorbent. The dechlorinated product may comprise alkylate gasoline, jet fuel, diesel fuel, base oil, or a combination thereof.

In another embodiment, the present invention further provides a hydrocarbon conversion and hydrodechlorination process comprising contacting at least one hydrocarbon reactant with an ionic liquid catalyst in a hydrocarbon conversion zone under hydrocarbon conversion conditions to provide used ionic liquid combined with conjunct polymer; regenerating at least a portion of the used ionic liquid in a catalyst regeneration zone to provide reactivated ionic liquid catalyst and free conjunct polymer; after the prior step, separating the conjunct polymer from the ionic liquid catalyst; and after the prior step, contacting the separated conjunct polymer with a hydrodechlorination catalyst in the presence of hydrogen in a hydrodechlorination zone under hydrodechlorination conditions to provide a dechlorinated product.

As used herein, the terms “comprising” and “comprises” mean the inclusion of named elements or steps that are identified following those terms, but not necessarily excluding other unnamed elements or steps.

### DETAILED DESCRIPTION

Ionic liquid catalysts may be useful for a range of hydrocarbon conversion processes, including paraffin alkylation, paraffin isomerization, olefin isomerization, olefin dimerization, olefin oligomerization, olefin polymerization and aromatic alkylation. Applicants have now discovered that products and by-products from ionic liquid catalyzed hydrocarbon conversion processes may be efficiently dechlorinated by contact with a hydrodechlorination catalyst in a hydrodechlorination zone in the presence of hydrogen at relatively low pressure, to provide HCl-containing off-gas and a dechlorinated product, wherein the chloride content of the dechlorinated product is low enough to allow the product to be used for blending into refinery products. Applicants have further discovered that the HCl may be recovered from the dechlorination off-gas, to provide HCl for recycling to the ionic liquid catalyzed hydrocarbon conversion process.

The term “Periodic Table” as referred to herein is the IUPAC version of the Periodic Table of the Elements dated Jun. 22, 2007, and the numbering scheme for the Periodic Table Groups is as described in Chemical and Engineering News, 63(5), 27 (1985).

### Ionic Liquid Catalysts

In an embodiment, processes according to the present invention may use a catalytic composition comprising at least one metal halide and at least one quaternary ammonium halide and/or at least one amine halohydride. The ionic liquid catalyst can be any halogen aluminate ionic liquid catalyst, e.g., comprising an alkyl substituted quaternary amine halide, an alkyl substituted pyridinium halide, or an alkyl substituted imidazolium halide of the general formula N⁺R₄X⁻. As an example, an ionic liquid useful in practicing the present invention may be represented by general formulas A and B,

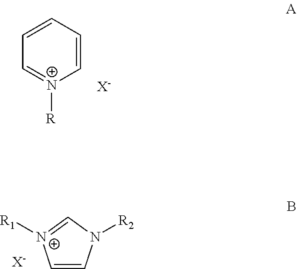

wherein R═H, methyl, ethyl, propyl, butyl, pentyl or hexyl, and X is a halide, and R₁ and R₂═H, methyl, ethyl, propyl, butyl, pentyl or hexyl, wherein R₁ and R₂ may or may not be the same. In an embodiment, X is chloride.

An exemplary metal halide that may be used in accordance with the present invention is aluminum chloride (AlCl₃). Quaternary ammonium halides which can be used in accordance with the present invention include those described in U.S. Pat. No. 5,750,455, the disclosure of which is incorporated by reference herein.

In an embodiment, the ionic liquid catalyst may be a chloroaluminate ionic liquid prepared by mixing AlCl₃ and an alkyl substituted pyridinium halide, an alkyl substituted imidazolium halide, a trialkylammonium hydrohalide, or a tetraalkylammonium halide, as disclosed in commonly assigned U.S. Pat. No. 7,495,144, the disclosure of which is incorporated by reference herein in its entirety.

In a sub-embodiment, the ionic liquid catalyst may comprise N-butylpyridinium heptachlorodialuminate ionic liquid, which may be prepared, for example, by combining AlCl₃with a salt of the general formula A, supra, wherein R is n-butyl and X is chloride. The present invention does not need to be limited to particular ionic liquid catalyst compositions.

### Feedstocks for Ionic Liquid Catalyzed Processes

In an embodiment, feeds for the present invention may comprise various streams in a petroleum refinery, a gas-to-liquid conversion plant, a coal-to-liquid conversion plant, or in naphtha crackers, middle distillate cracker or wax crackers, FCC offgas, FCC light naphtha, coker offgas, coker naphtha, hydrocracker naphtha, and the like. Such streams may contain isoparaffin(s) and/or olefin(s). Such streams may be fed to the reactor of a hydrocarbon conversion system of the present invention via one or more feed dryer units (not shown).

Examples of olefin containing streams include FCC offgas, coker gas, olefin metathesis unit offgas, polyolefin gasoline unit offgas, methanol to olefin unit offgas, FCC light naphtha, coker light naphtha, Fischer-Tropsch unit condensate, and cracked naphtha. Some olefin containing streams may contain two or more olefins selected from ethylene, propylene, butylenes, pentenes, and up to C₁₀ olefins.

The olefin containing stream can be a fairly pure olefinic hydrocarbon cut or can be a mixture of hydrocarbons having different chain lengths thus a wide boiling range. The olefinic hydrocarbon can be terminal olefin (an alpha olefin) or can be an internal olefin (having an internal double bond). The olefinic hydrocarbon chain can be either straight chain or branched or a mixture of both. In one embodiment of the present invention, the olefinic feed may comprise a mixture of mostly linear olefins from C₂ to about C₃₀. The olefins may be mostly, but not entirely, alpha olefins. In another embodiment of the present invention, the olefinic feed can comprise 50% of a single alpha olefin species. In another embodiment of the present invention, the olefinic feed can comprise at least 20% of alpha olefin species.

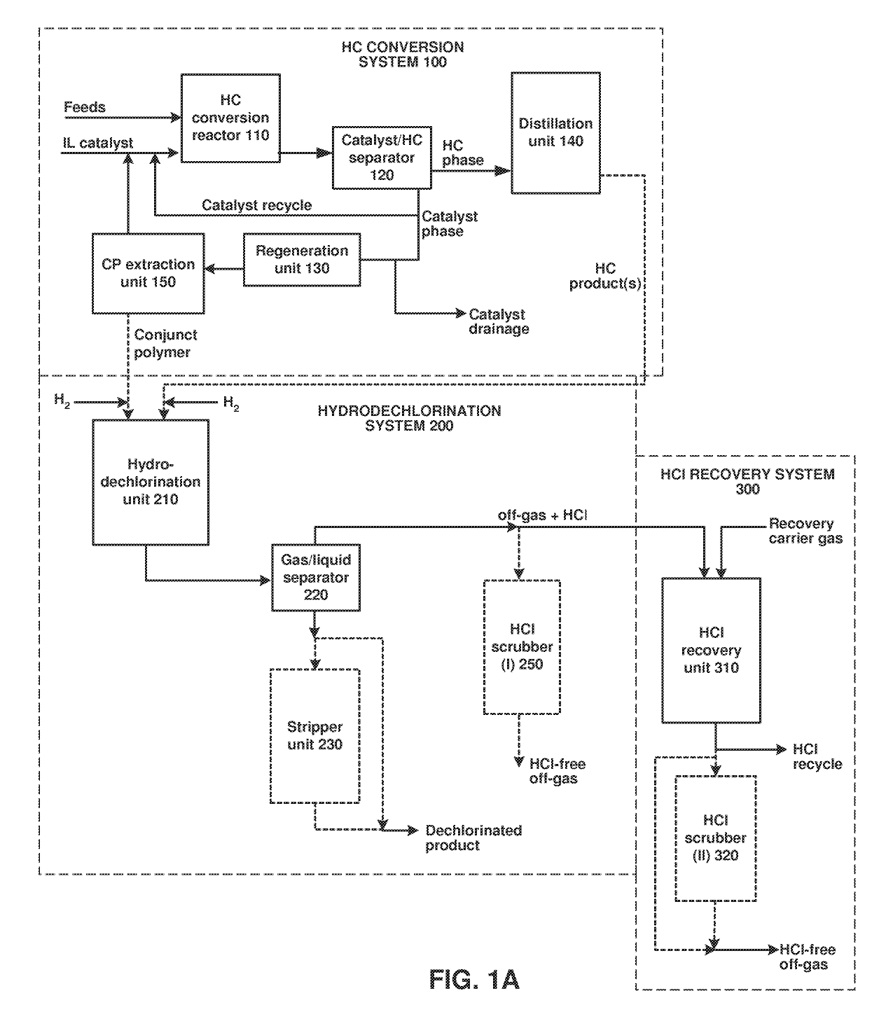

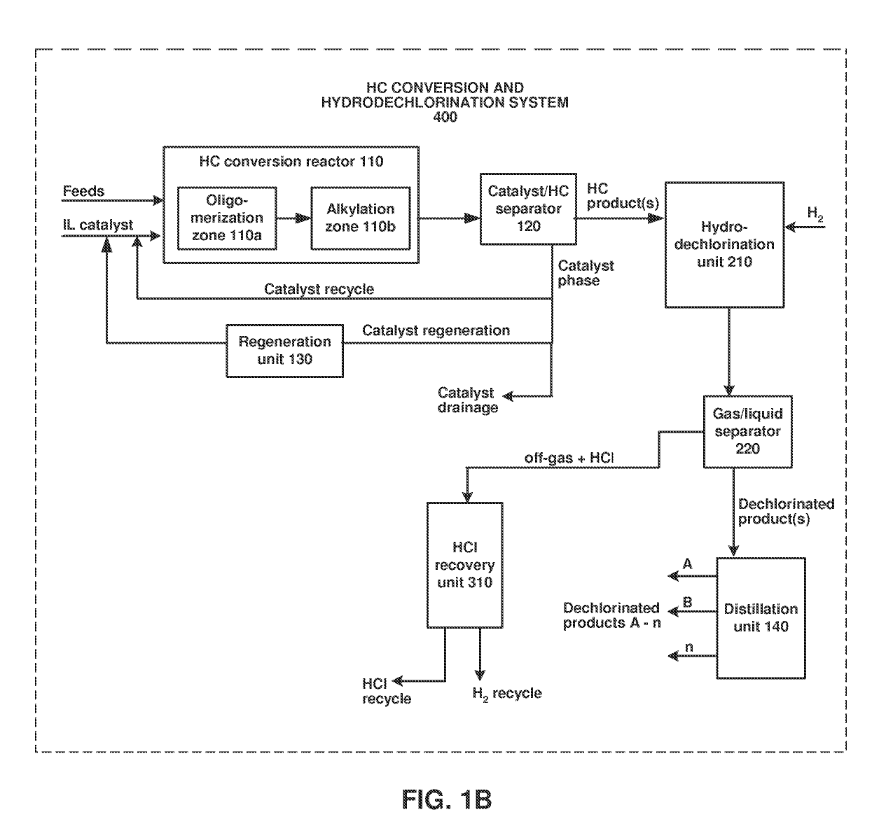

In an embodiment, olefins in the feed may also undergo oligomerization when contacted with an ionic liquid catalyst in the hydrocarbon conversion reactor. Ionic liquid catalyzed olefin oligomerization may take place under the same or similar conditions as ionic liquid catalyzed olefin-isoparaffin alkylation. As a result, in an embodiment of the present invention, both olefin oligomerization and olefin-isoparaffin alkylation may take place in a single reaction zone of the hydrocarbon conversion reactor. In an embodiment, olefin oligomerization and olefin-isoparaffin alkylation may take place in an oligomerization zone 110a and an alkylation zone 110b, respectively, of hydrocarbon conversion reactor 110 (see, for example, FIG. 1B). In an embodiment of the present invention, an oligomeric olefin produced in oligomerization zone 110a may be subsequently alkylated by reaction with an isoparaffin in alkylation zone 110b to provide a distillate, and/or lubricant component or base oil product. Ionic liquid catalyzed olefin oligomerization and olefin-isoparaffin alkylation is disclosed, for example, in commonly assigned U.S. Pat. Nos. 7,572,943 and 7,576,252 both to Elomari et al., the disclosures of which are incorporated by reference herein in their entirety.

Examples of isoparaffin containing streams include, but are not limited to, FCC naphtha, hydrocracker naphtha, coker naphtha, Fisher-Tropsch unit condensate, and cracked naphtha. Such streams may comprise a mixture of two or more isoparaffins. In a sub-embodiment, a feed for an ionic liquid catalyzed process of the invention may comprise isobutane, which may be obtained, for example, from a hydrocracking unit or may be purchased.

### Reaction Conditions for Ionic Liquid Catalyzed Hydrocarbon Conversions

Due to the low solubility of hydrocarbons in ionic liquids, hydrocarbon conversion reactions in ionic liquids (including isoparaffin-olefin alkylation reactions) are generally biphasic and occur at the interface in the liquid state. The volume of ionic liquid catalyst in the reactor may be generally in the range from about 1 to 70 vol %, and usually from about 4 to 50 vol %. Generally, vigorous mixing (e.g., stirring or Venturi nozzle dispensing) is used to ensure good contact between the reactants and the ionic liquid catalyst. The reaction temperature may be generally in the range from about −40° F. (about −40 degree Celsius) to +480° F. (248.9 degree Celsius), typically from about −4° F. (−20 degree Celsius) to +210° F. (98.89 degree Celsius), and often from about +40° F. (4.444 degree Celsius) to +140° F. (60 degree Celsius). The reactor pressure may be in the range from atmospheric pressure to about 8000 kPa. Typically, the reactor pressure is sufficient to keep the reactants in the liquid phase.

Residence time of reactants in the reactor may generally be in the range from a few seconds to hours, and usually from about 0.5 min to 60 min. In the case of ionic liquid catalyzed isoparaffin-olefin alkylation, the reactants may be introduced in an isoparaffin:olefin molar ratio generally in the range from about 1 to 100, more typically from about 2 to 50, and often from about 2 to 20. Heat generated by the reaction may be dissipated using various means well known to the skilled artisan.

In the case of olefin oligomerization, e.g., in oligomerization zone 110a (FIG. 1B), oligomerization conditions for the present invention may include a temperature in the range from about 30° F. (about −1.1 degree Celsius) to about 300° F. (about 148.9 degree Celsius), typically from about 30° F. (about −1.1 degree Celsius) to about 210° F. (about 98.89 degree Celsius), and usually from about 30° F. (about −1.1 degree Celsius) to about 120° F. (about 48.89 degree Celsius).

### Ionic Liquid Catalyzed Hydrocarbon Conversion Systems and Processes

In FIG. 1A, systems within the scheme may be represented by em dash lines (———); while optional or alternative lines, conduits, units, or steps may be represented by en dash lines (———). With reference to FIG. 1A, an ionic liquid catalyzed hydrocarbon conversion system 100 according to an embodiment of the present invention may include a hydrocarbon conversion reactor 110, a catalyst/hydrocarbon separator 120, a catalyst regeneration unit 130, a distillation unit 140, and a conjunct polymer (CP) extraction unit 150.

During an ionic liquid catalyzed hydrocarbon conversion process of the instant invention, dry feeds may be introduced into hydrocarbon conversion reactor 110. Hydrocarbon conversion reactor 110 may also be referred to herein as a hydrocarbon conversion zone. The dry feeds may include at least one hydrocarbon reactant, which may be introduced into hydrocarbon conversion reactor 110 via one or more reactor inlet ports (not shown). In an embodiment, the at least one hydrocarbon reactant may comprise a first reactant comprising a C₄-C₁₀ isoparaffin and a second reactant comprising a C₂-C₁₀ olefin.

Ionic liquid catalyst may be introduced into hydrocarbon conversion reactor 110 via a separate inlet port (not shown). The feeds to hydrocarbon conversion reactor 110 may further include a catalyst promoter, such as anhydrous HCl or an alkyl halide. In an embodiment, the catalyst promoter may comprise a C₂-C₆ alkyl chloride. In a sub-embodiment, the catalyst promoter may comprise n-butyl chloride or t-butyl chloride. Hydrocarbon conversion reactor 110 may be vigorously mixed to promote contact between reactant(s) and ionic liquid catalyst. Reactor conditions may be adjusted to optimize process performance for a particular hydrocarbon conversion process of the present invention.

During hydrocarbon conversion processes of the invention, hydrocarbon conversion reactor 110 may contain a mixture comprising ionic liquid catalyst and a hydrocarbon phase. The hydrocarbon phase may comprise at least one hydrocarbon product of the ionic liquid catalyzed reaction. The ionic liquid catalyst may be separated from the hydrocarbon phase via catalyst/hydrocarbon separator 120, wherein the hydrocarbon and ionic liquid catalyst phases may be allowed to settle under gravity, by using a coalescer, or by a combination thereof. The use of coalescers for liquid-liquid separations is described in US Publication Number 20100130800A1, the disclosure of which is incorporated by reference herein in its entirety. The hydrocarbon phase may be fed from catalyst/hydrocarbon separator 120 to distillation unit 140. At least a portion of the ionic liquid phase may be recycled directly to hydrocarbon conversion reactor 110.

With continued operation of hydrocarbon conversion system 100, the ionic liquid catalyst may become partially deactivated or spent. Catalyst deactivation is associated with the formation of conjunct polymer in the ionic liquid phase, for example, as disclosed in commonly assigned U.S. Pat. No. 7,674,739, the disclosure of which is incorporated by reference herein in its entirety. In order to maintain the catalytic activity, at least a portion of the ionic liquid phase may be fed to catalyst regeneration unit 130 for regeneration of the ionic liquid catalyst. In an embodiment, the portion of the ionic liquid phase fed to catalyst regeneration unit 130 may be generally in the range from about 1% to 95%, and typically from about 5% to 75%.

In an embodiment, the ionic liquid catalyst may be regenerated by treatment with a regeneration metal. As an example, a process for the regeneration of ionic liquid catalyst by treatment with Al metal is disclosed in U.S. Pat. No. 7,674,739, incorporated by reference herein. In another embodiment, the ionic liquid may be regenerated by treatment, in the presence of H₂, with a hydrogenation catalyst (see, for example, U.S. Pat. No. 7,691,771 to Harris et al., the disclosure of which is incorporated by reference herein in its entirety).

In an embodiment of the present invention, fresh ionic liquid catalyst may be introduced into hydrocarbon conversion reactor 110 during a hydrocarbon conversion process. The catalytic activity of hydrocarbon conversion reactor 110 may be maintained under steady state conditions by monitoring the catalytic activity, and by adjusting process parameters, such as the degree of catalyst regeneration, the amount of catalyst drainage, the amount of fresh ionic liquid introduced, and combinations thereof, according to the monitored catalytic activity. The catalytic activity may be gauged, for example, by monitoring the concentration of conjunct polymer in the ionic liquid catalyst phase.

The conjunct polymer that has combined with the used ionic liquid may be released from the ionic liquid during ionic liquid catalyst regeneration. The free conjunct polymer may then be separated from the regenerated ionic liquid catalyst in a conjunct polymer (CP) extraction unit 150. The conjunct polymer may be extracted from the used ionic liquid, e.g., using a C₄-C₁₅ hydrocarbon (e.g., alkane), and typically a C₄-C₁₀ alkane, such as isobutane or alkylate gasoline. The regenerated ionic liquid catalyst may be fed from the conjunct polymer extraction unit 150 to hydrocarbon conversion reactor 110.

The hydrocarbon phase from catalyst/hydrocarbon separator 120 may be fed to distillation unit 140. Distillation unit 140 may represent or comprise a plurality of distillation columns. In an embodiment, distillation unit 140 may comprise one (1), two (2), three (3), four (4), or more distillation columns. Distillation unit 140 may be adjusted, e.g., with respect to temperature and pressure, to provide at least one hydrocarbon product from the hydrocarbon phase under steady state distillation conditions.

In an embodiment of the present invention, a hydrocarbon product obtained from distillation unit 140 may comprise at least one halogenated component. As an example only, the hydrocarbon product may have an organic chloride content generally greater than about 50 ppm, typically greater than 100 ppm, and often greater than 200 ppm. In an embodiment, a hydrocarbon product from distillation unit 140 may have an organic chloride content generally in the range from about 50 ppm to 5000 ppm, typically from about 100 ppm to 4000 ppm, and often from about 200 ppm to 3000 ppm.

The hydrocarbon product(s), which may include at least one halogenated component, may be fed, e.g., from distillation unit 140 to hydrodechlorination unit 210 for hydrodechlorinating the hydrocarbon product(s) by contacting the at least one hydrocarbon product with a hydrodechlorination catalyst in the presence of hydrogen in a hydrodechlorination zone under hydrodechlorination conditions to provide: i) at least one dechlorinated product and ii) an off-gas comprising HCl, as described herein below. In general, a first chloride content of the at least one hydrocarbon product prior to hydrodechlorination according to the present invention is greater than 50 ppm, and typically much greater than 50 ppm; while after hydrodechlorination according to the present invention, a second chloride content of the dechlorinated product(s) is less than 50 ppm, and typically less than about 10 ppm.

With reference to FIG. 1B, an ionic liquid catalyzed hydrocarbon conversion and hydrodechlorination system 400 according to another embodiment of the present invention may include a hydrocarbon conversion reactor 110, a catalyst/hydrocarbon separator 120, a hydrodechlorination unit 210, a catalyst regeneration unit 130, a gas/liquid separator 220, an HCl recovery unit 310, and a distillation unit 140.

During an ionic liquid catalyzed hydrocarbon conversion process of the instant invention, dry feeds may be introduced into hydrocarbon conversion reactor 110. Hydrocarbon conversion reactor 110 may also be referred to herein as a hydrocarbon conversion zone. In an embodiment, hydrocarbon conversion reactor 110 may include an oligomerization zone 110a and an alkylation zone 110b. The dry feeds may include at least one hydrocarbon reactant, e.g., substantially as described herein above with reference to FIG. 1A. Reaction conditions for ionic liquid catalyzed hydrocarbon conversions are described herein above. Reactor conditions within each of oligomerization zone 110a and alkylation zone 110b may be adjusted to optimize process performance, e.g., for particular hydrocarbon feeds or desired products.

Hydrocarbon product(s) from hydrocarbon conversion reactor 110 may be separated from the ionic liquid via catalyst/hydrocarbon separator 120, as described with reference to FIG. 1A. The hydrocarbon product(s), which may include at least one chlorinated component, may be fed, e.g., from catalyst/hydrocarbon separator 120 to hydrodechlorination unit 210 for hydrodechlorinating the hydrocarbon product(s). Such hydrodechlorination may be performed by contacting the at least one hydrocarbon product with a hydrodechlorination catalyst in the presence of hydrogen in a hydrodechlorination zone under hydrodechlorination conditions to provide: i) at least one dechlorinated product and ii) an off-gas comprising HCl, as described herein below. In general, a first chloride content of the at least one hydrocarbon product prior to hydrodechlorination according to the present invention is greater than 50 ppm, and typically much greater than 50 ppm. In contrast, as a result of hydrodechlorination according to the present invention, a second chloride content of the dechlorinated product(s) is less than 50 ppm, and typically less than about 10 ppm.

### Hydrodechlorination of Ionic Liquid Catalyzed Hydrocarbon Conversion Products

With further reference to FIGS. 1A and 1B, at least one hydrocarbon product, e.g., derived from an ionic liquid catalyzed alkylation reaction, may be fed together with hydrogen into a hydrodechlorination unit 210. In an embodiment, the at least one hydrocarbon product may comprise a distilled hydrocarbon product from distillation unit 140 (see, e.g., FIG. 1A). In an embodiment, the at least one hydrocarbon product may comprise alkylate gasoline, diesel fuel, jet fuel, base oil, or a combination thereof.

In another embodiment of the present invention, the at least one hydrocarbon product may comprise a plurality of hydrocarbon products, which may be fed to hydrodechlorination unit 210, e.g., en masse, from catalyst/hydrocarbon separator 120 before undergoing fractionation (see, for example, FIG. 1B).

Hydrodechlorination unit 210 may contain a hydrodechlorination catalyst. The hydrodechlorination unit 210 may also be referred to herein as a hydrodechlorination zone. The hydrodechlorination catalyst may comprise an element selected from elements of Groups 6, 8, 9, 10, and 11 of the Periodic Table, and their mixtures, present as metals, oxides, or sulfides. In a sub-embodiment, the hydrodechlorination catalyst may comprise an element selected from Pd, Pt, Au, Fe, Ni, Co, Mo, and W, and their mixtures, present as metals, oxides, or sulfides.

The hydrodechlorination catalyst may further comprise a support. The support may comprise an inorganic porous material, such as a refractory oxide, or an activated carbon. Examples of refractory oxide support materials include alumina, silica, titania, alumina-silica, and zirconia, or the like, and combinations thereof. In an embodiment, the hydrodechlorination catalyst may comprise a noble metal on a refractory oxide support. In a sub-embodiment, the hydrodechlorination catalyst may comprise Pd, e.g., in the range from about 0.05 to 3.0 wt % Pd.

Within hydrodechlorination unit 210, the at least one hydrocarbon product may be contacted with the hydrodechlorination catalyst in the presence of hydrogen under hydrodechlorination conditions to provide: i) a dechlorinated product and ii) an off-gas comprising HCl. In an embodiment, the dechlorinated product may comprise dechlorinated alkylate gasoline, dechlorinated jet fuel, dechlorinated diesel fuel, or dechlorinated base oil. The dechlorinated product may be separated from the off-gas via a gas/liquid separator 220. The hydrodechlorination system 200 upstream from gas/liquid separator 220 may be above ambient pressure, and gas/liquid separator 220 may also be referred to herein as a high pressure separator.

In an embodiment, gas/liquid separator 220 may be operated at a temperature generally in the range from about 50° F. (about 10 degree Celsius) to 600° F. (315.6 degree Celsius), typically from about 100° F. (about 37.78 degree Celsius) to 550° F. (287.8 degree Celsius), and often from about 100° F. (about 37.78 degree Celsius) to 500° F. (260 degree Celsius). In an embodiment, gas/liquid separator 220 may be operated at a maximum liquid level typically not more than about 85%, usually not more than about 75%, and often not more than about 65% of the total height or volume of gas/liquid separator 220. As a non-limiting example, a major portion of the HCl can be constrained in the gas phase, for subsequent recovery therefrom, when gas/liquid separator 220 is operated at a suitable temperature within the range cited hereinabove and at a liquid level equal to or less than about 65%.

The hydrodechlorination conditions within the hydrodechlorination zone may comprise a reaction temperature generally in the range from about 300° F. (about 148.9 degree Celsius) to 750° F. (398.9 degree Celsius), and typically from about 400° F. (about 204.4 degree Celsius) to 650° F. (343.3 degree Celsius). The hydrodechlorination conditions may include a reaction pressure generally in the range from about 100 to 5000 psig, and typically from about 200 to 2000 psig. A liquid hourly space velocity (LHSV) feed rate to the hydrodechlorination zone may be generally in the range from about 0.1 to 50, and typically from about 0.2 to 10. A hydrogen supply to the hydrodechlorination zone may be generally in the range from about 50 to 8000 standard cubic feet per barrel (SCFB) of hydrocarbon product, and typically from about 100 to 5000 SCFB.

The hydrocarbon product feed to hydrodechlorination unit 210 may typically have a much higher chloride content as compared with that of the dechlorinated product obtained from hydrodechlorination unit 210. In an embodiment, a first chloride content of at least one hydrocarbon product fed to hydrodechlorination unit 210 may be greater than about 50 ppm. In an embodiment, the hydrocarbon product feed to hydrodechlorination unit 210 may have an organic chloride content generally in the range from about 50 ppm to 5000 ppm, typically from about 100 ppm to 4000 ppm, and often from about 200 ppm to 3000 ppm. In contrast, the chloride content of the dechlorinated product is lower than that of the feed, typically less than 50 ppm, and usually less than about 10 ppm.

With further reference to hydrodechlorination system 200 of FIG. 1A, in an embodiment the dechlorinated product obtained from gas/liquid separator 220 may comprise alkylate gasoline, having similar or substantially the same octane number and boiling point distribution as compared with the alkylate feed, while the chloride content is greatly decreased. Typically, a dechlorinated product, such as alkylate gasoline, provided by processes of the present invention may have a chloride content less than 50 ppm, and often equal to or less than about 10 ppm. Analogous results will be obtained when the present invention is practiced using catalyst systems based on halides other than chlorides.

In an embodiment, the dechlorinated product may be fed to a stripper unit 230 for removing any residual off-gas components. As an example, such stripping may be performed using a counter-current stream of dry nitrogen gas. In an embodiment wherein gas/liquid separator 220 is operated under suitable temperature and other conditions, the dechlorinated product from gas/liquid separator 220 may have a chloride content (e.g., <10 ppm chloride) and other specifications well within acceptable ranges, and therefore a stripping procedure may optionally be omitted.

The off-gas produced by hydrodechlorination unit 210 may comprise substantial amounts of H₂, in addition to HCl. The off-gas produced in hydrodechlorination unit 210 may further comprise from about 0.1 to 20 vol % C₁-C₅ hydrocarbons. The off-gas produced in hydrodechlorination unit 210 may still further comprise C₅₊ hydrocarbons.

The off-gas from hydrodechlorination unit 210 may be fed to an HCl recovery system 300 (see, e.g., FIG. 1A) for removing the HCl from the off-gas and for recovering the HCl, as described herein below. The off-gas from hydrodechlorination unit 210 may be fed to an HCl scrubber 250 for HCl removal from the off-gas. Then the HCl-free off-gas, which may comprise predominantly H₂ gas, can be recycled back to hydrodechlorination unit 210.

### Hydrodechlorination of Conjunct Polymer Feed

While not being bound by any theory, the formation of by-products known as conjunct polymer during ionic liquid catalyzed hydrocarbon conversion reactions can be associated with ionic liquid catalyst deactivation. Conjunct polymer may comprise a mixture of polyunsaturated acyclic, cyclic, and polycyclic molecules that may include one or a combination of 4-, 5-, 6- and 7-membered rings in their skeletons. Some examples of the likely polymeric species were reported by Miron et al. (Journal of chemical and Engineering Data, 1963) and Pines (Chem. Tech, 1982). The accumulation of conjunct polymer can deactivate chloroaluminate ionic liquid catalysts by weakening the acid strength of the catalyst through the formation of complexes of conjunct polymers with AlCl₃.

Applicants have now discovered that conjunct polymer, e.g., that may be released during catalyst regeneration, may provide a valuable feedstock for liquid fuel production processes. In an embodiment, used ionic liquid catalyst may be regenerated by treatment with a regeneration metal. The regeneration metal may be, e.g., Al, Ga, In, and Zn. The metals may be in the form of fine particles, granules, sponges, gauzes, etc. An effective amount of metal, say aluminum, used for the regeneration of used ionic liquid catalyst may be determined by the amount (concentration) of conjunct polymer in the used ionic liquid.

The particular regeneration metal to be used may be selected based on the composition of the ionic liquid catalyst, e.g., to prevent the contamination of the catalyst with unwanted metal complexes or intermediates that may form and remain in the catalyst phase. As an example, aluminum metal will be the metal of choice for the regeneration when the catalyst system is a chloroaluminate ionic liquid-based catalyst.

With further reference to FIG. 1A, the regenerated ionic liquid may be sent to conjunct polymer extraction unit 150, in which free conjunct polymer that is released from the ionic liquid during catalyst regeneration may be extracted with a hydrocarbon, e.g., a C₃-C₁₀ alkane. In an embodiment, the hydrocarbon solvent used for extracting the conjunct polymer may comprise isobutane. After phase separation, the organic phase may be sent to a stripper to separate the extracted conjunct polymer from the solvent. A process for ionic liquid catalyst regeneration in which released conjunct polymer is separated from the catalyst phase, is disclosed in commonly assigned U.S. Pat. No. 7,732,364, the disclosure of which is incorporated by reference herein in its entirety.

A conjunct polymer feed, e.g., obtained from conjunct polymer extraction unit 150, may have an organic chloride content generally in the range from about 50 ppm to 5000 ppm, typically from about 100 ppm to 4000 ppm, and often from about 200 ppm to 3000 ppm. The conjunct polymer feed, may be dechlorinated substantially as described hereinabove for the dechlorination of alkylate distillate. In an embodiment, a first chloride content of the conjunct polymer feed may greater than about 50 ppm or greater, and the chloride content of the dechlorinated product is lower than that of the feed, generally the chloride content of the dechlorinated product being less than 50 ppm, and typically the chloride content of the dechlorinated product being less than 10 ppm.

When using conjunct polymer as feed to hydrodechlorination unit 210, at least about 90% of the dechlorinated product derived from the conjunct polymer feed may have a boiling point range generally from about 200° F. (about 93.33 degree Celsius) to 1000° F. (537.8 degree Celsius), and often from about 200° F. (about 93.33 degree Celsius) to 800° F. (426.7 degree Celsius). In an embodiment, the dechlorinated product may comprise base oil, or a middle distillate fuel, such as jet fuel or diesel fuel, wherein the dechlorinated product may have a chloride content generally less than about 50 ppm, and more typically less than about 10 ppm.

### HCl Capture, Recovery, and Recycle

According to an aspect of the present invention, the off-gas from hydrodechlorination unit 210 may be fed from gas/liquid separator 220 to HCl recovery system 300 for removing the HCl from the off-gas and for recovering the HCl. The off-gas may be fed through HCl recovery unit 310 to capture the HCl. The off-gas may comprise H₂ and C₁-C₅ hydrocarbons in addition to HCl.

HCl recovery unit 310 may contain an adsorbent for adsorbing the HCl present in the off-gas. The HCl recovery unit 310 may also be referred to herein as an HCl adsorption zone. The off-gas may be contacted with the adsorbent under HCl adsorbing conditions such that the HCl is adsorbed by the adsorbent. In an embodiment, the off-gas may be fed through HCl recovery unit 310 at about ambient temperature and a pressure in the range from about atmospheric pressure to the pressure of the gas/liquid separator 220 to capture the HCl. The adsorbent may be selective, such that HCl is selectively adsorbed, while H₂ and light hydrocarbons flow through the absorbent to provide HCl-free off-gas.

The adsorbent within HCl recovery unit 310 may comprise a material selected from a molecular sieve, a refractory oxide, an activated carbon, or combinations thereof. In an embodiment, the adsorbent may comprise a refractory oxide selected from alumina, silica, titania, silica-alumina, and zirconia, or the like, and combinations thereof. In an embodiment, the adsorbent may comprise a molecular sieve, including 8-, 10-, and 12-ring zeolites, and combinations thereof, wherein the zeolites may have a Si/Al ratio in the range from 1 to ∞. Some examples of molecular sieves that may be used as adsorbents in practicing the present invention include the following: 3A, 4A, 5A, 13X, 13Y, USY, ZSM-5, ZSM-22, ZSM-23, ZSM-35, ZSM-48, MCM-22, MCM-35, MCM-58, SAPO-5, SAPO-11, SAPO-35, and VPI-5. In a sub-embodiment, the adsorbent may comprise zeolite 4A. In another sub-embodiment, the adsorbent may comprise zeolite 13X. Zeolites and molecular sieves are well known in the art (see, for example, Zeolites in Industrial Separation and Catalysis, By Santi Kulprathipanja, Pub. Wiley-VCH, 2010).

In an embodiment, HCl recovery unit 310 may include two adsorption beds (not shown), which may be arranged in parallel to facilitate the HCl adsorption/desorption cycles. The feed to HCl recovery unit 310 may be controlled, e.g., via a valve, whereby after the first adsorbent bed is saturated with HCl from the off-gas, the flow of off-gas to HCl recovery unit 310 can be turned to the second adsorbent bed. The adsorbed HCl on the first adsorbent bed may be recovered from the adsorbent, e.g., by feeding a recovery carrier gas through the spent adsorbent bed. In an embodiment, the recovery carrier gas may comprise dry N₂. In another embodiment, the recovery carrier gas may comprise a C₃-C₈ alkane, such as isobutane. Desorption of the HCl from the adsorbent may be completed at the ambient temperature and the system pressure, or may be promoted by heating the adsorbent via the recovery carrier gas, or by operating the HCl recovery unit 310 at a pressure lower than the adsorption pressure. In an embodiment, the adsorbent may be heated to a temperature in the range from about 100° F. (about 37.78 degree Celsius) to 1000° F. (537.8 degree Celsius), and typically from about 200° F. (about 93.33 degree Celsius) to 800° F. (426.7 degree Celsius) to promote desorption of the HCl from the adsorbent. The desorption pressure may be generally in the range from about 0 to 500 psig, and typically from about 20 to 300 psig. In an embodiment, the HCl recovered from the adsorbent may be recycled to hydrocarbon conversion reactor 110 (see, e.g., FIGS. 1A and 1B). Since HCl serves as a promoter of ionic liquid catalyzed hydrocarbon conversion reactions, the required amount of fresh HCl or organic halide promoter is thereby decreased, thus providing a substantial economic benefit to the overall hydrocarbon conversion process of the invention.

Due to the presence of HCl in the hydrocarbon conversion, hydrodechlorination, and HCl recovery systems of the present invention, processes of the present invention may be performed entirely under anhydrous conditions.

HCl recovery system 300 may further include an HCl scrubber 320, such that the off-gas may be fed to HCl scrubber 320 for HCl removal from the off-gas. As an example, HCl scrubber 320 may serve as a contingency or back-up capability, e.g., in the event that HCl recovery unit 310 may be temporarily inoperative or unavailable. In an embodiment, HCl recovery system 300 may include one or more additional HCl recovery units (not shown), one or more of which may be operated in parallel with HCl recovery unit 310, whereby a first HCl recovery unit may be operated in adsorption mode while a second HCl recovery unit may be operated in desorption mode. The HCl-free off-gas from HCl recovery system 300, which may comprise mostly H₂ gas, may be recycled back to the hydrodechlorination unit 210 to minimize the consumption of H₂.

The following examples are illustrative of the present invention, but are not intended to limit the invention in any way beyond what is contained in the claims which follow.

### EXAMPLES

### Example 1

### Ionic Liquid Catalyst Comprising Anhydrous Metal Halide

Various ionic liquid catalysts comprising metal halides such as AlCl₃, AlBr₃, GaCl₃, GaBr₃, InCl₃, and InBr₃ may be used for practicing the catalytic processes of the present invention. N-butylpyridinium heptachlorodialuminate (C₅H₅NC₄H₉Al₂Cl₇) ionic liquid catalyst is an example of such a catalyst. The catalyst has the following composition.

| | |
|---|---|
| Wt % Al | 12.4 |
| Wt % Cl | 56.5 |
| Wt % C | 24.6 |
| Wt % H | 3.2 |
| Wt % N | 3.3 |

N-butylpyridinium heptachlorodialuminate may be prepared, e.g., according to Example 1 of U.S. Pat. No. 7,432,408, or may be purchased (Alfa Aesar, Ward Hill, Mass.).

### Example 2

### Preparation of Alkylate Distillate

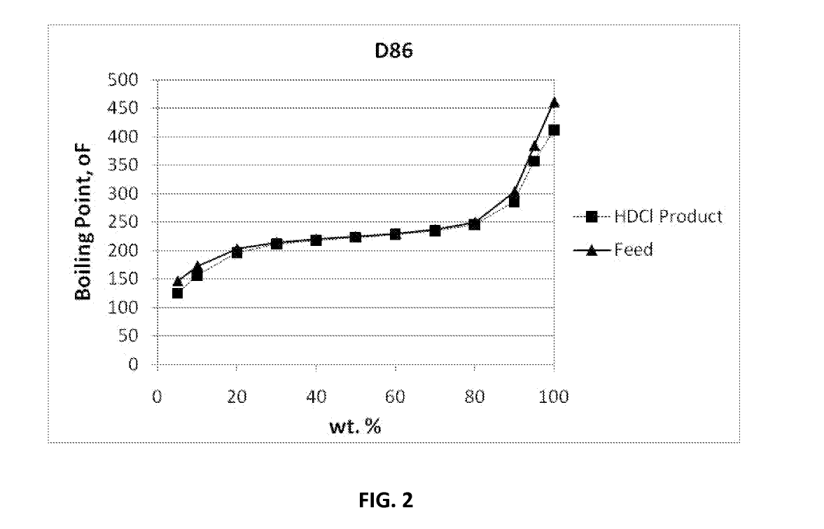

A chlorinated alkylate was prepared by reacting isobutane with C₃-C₄ olefins at an isobutane to olefin molar ratio of 8 in the presence of N-butylpyridinium heptachlorodialuminate (6 vol %) as catalyst and n-butyl chloride as catalyst promoter. The alkylation reaction was conducted at 95° F. (35 degree Celsius) and 190 psig with vigorous stirring. After phase separation, the hydrocarbon phase provided a chlorinated alkylate (“feed”) having the boiling point distribution as shown in FIG. 2, and a C₈ composition as shown in Table 1.

### Example 3

### Preparation of Hydrodechlorinated Alkylate Product

The alkylate prepared according to Example 2 was hydrodechlorinated over a Pd/alumina catalyst containing 0.5 wt % Pd, as follows. The hydrodechlorination catalyst was first reduced in flowing hydrogen at 450° F. (232.2 degree Celsius), 500 psig for two hours. Then, hydrodechlorination of the alkylate prepared according to Example 2 was performed at an average catalyst temperature of 500° F. (260 degree Celsius), a pressure of 500 psig, a LHSV of 1.0 hr⁻¹, and a H₂ feed rate of 1000 SCFB.

The hydrodechlorinated alkylate product had the boiling characteristics as shown in FIG. 2. It can be seen that hydrodechlorination according to the present invention did not substantially alter the boiling characteristics of the alkylate “feed” prepared according to Example 2.

### Example 4

### C₈ Composition of Alkylate Feed and Hydrodechlorination Whole Liquid Product

The alkylate feed of Example 2 and the dechlorinated whole liquid product obtained using the hydrodechlorination procedure of Example 3 were each subjected to 0₈ composition analysis by GC, and the results are shown in Table 1. The dechlorinated product had a trimethylpentane content of about 83.3% and a trimethylpentane to dimethylhexane (TMP/DMH) ratio of about 5.32. These values are comparable to those for the alkylate feed: 83.5% and 5.39, respectively (Table 1).

TABLE 1
Comparison of C₈ composition for alkylate feed and dechlorinated product

| | Alkylate | Dechlorinated |
|---|---|---|
| | Feed | product^{†} |
| C₈ composition | | |
| C₈ in WLP, % | 60.5 | 61.7 |
| TMP/DMH | 5.39 | 5.32 |
| TMP in C₈, % | 83.5 | 83.3 |
| Chloride content (ppm) | 4048 | <3 |

^{†}Hydrodechlorination conditions as for Example 3; liquid product recovery was >95%.

It can be seen that hydrodechlorination according to the present invention did not substantially alter the percent of trimethylpentane in the total C₈ hydrocarbon fraction, nor the trimethylpentane to dimethylhexane (TMP/DMH) ratio of the dechlorinated product, as compared with the alkylate feed prepared according to Example 2.

### Example 5

### Quantitative Analysis of Alkylate Feed and Hydrodechlorinated Whole Liquid Product for Organic Chloride

The chloride content of the alkylate prepared according to Example 2 and of the hydrodechlorinated whole liquid product (Example 3) was determined using a bench-top XOS Clora chloride analyzer (X-Ray Optical Systems, Inc., East Greenbush, N.Y.). It can be seen from Table 1 that following hydrodechlorination the chloride content was decreased to <10 ppm.

### Example 6

### Preparation of Conjunct Polymer

A chlorinated conjunct polymer was prepared by regenerating a deactivated ionic liquid catalyst with aluminum metal followed by extraction with isobutane. The conjunct polymer was separated from the organic phase by distillation.

### Example 7

### Preparation of Hydrodechlorinated Conjunct Polymer

The conjunct polymer prepared according to Example 6 was hydrodechlorinated over a Pd/alumina catalyst containing 0.5 wt % Pd under the following conditions: 500° F. average catalyst bed temperature, 450 psig total pressure, 1.0 LHSV, and 3000 SCF/B.

Table 2 shows that hydrodechlorination process can significantly remove chloride impurity from the feed, by decreasing the chloride content from 301 ppm in the conjunct polymer feed to 2.9 ppm in the dechlorinated product. The hydrodechlorination process also hydrogenates unsaturated components in the conjunct polymer as indicated by the reduction of bromine number from 179 g-Br/100 g conjunct polymer of the feed to <1g-Br/100g of the dechlorinated product, thus improving diesel properties such as API and cetane index. The hydrodechlorination process also lowers the sulfur content of the conjunct polymer. The sulfur content in the conjunct polymer was reduced from 29.7 ppm to 7.8 ppm.

TABLE 2
Comparison of conjunct polymer feed and dechlorinated product

| | Conjunct Polymer | Dechlorinated |
|---|---|---|
| Feed ID | Feed | Product |
| API gravity | 34.1 | 35.4 |
| S, wt ppm | 29.7 | 7.8 |
| Bromine number, g-Br/100 g | 179 | <1 |
| Cl, ppm | 301 | 2.9 |
| Simdist, ° F. | | |
| 0.5 wt % | 226 | 212 |
| 10 wt % | 342 | 340 |
| 50 wt % | 492 | 500 |
| 90 wt % | 703 | 723 |
| 99.5 wt % | 953 | 988 |

### Example 8

### HCl Capture from Hydrodechlorination Processes

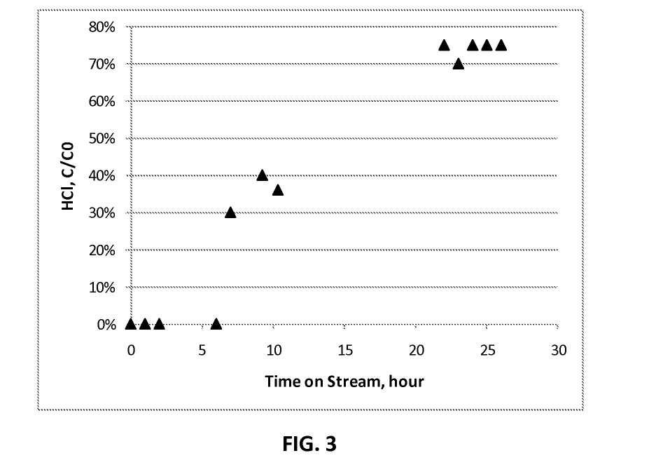

A HCl-containing off-gas from a hydrodechlorination process using alkylate distillate feed was fed directly to a HCl recovery unit (see, e.g., FIG. 1A) using zeolite 4A as adsorbent at a temperature of 100° F. The HCl concentration in the off-gas before and after contacting with adsorbent was periodically monitored and measured by HCl-selective Draeger tubes. FIG. 3 shows the HCl concentration measured in the off-gas as the % of the feed HCl concentration as a function of time. It can be seen from FIG. 3 that the HCl in the off-gas was selectively removed by the absorbent for about 7 hours. HCl breakthrough occurred at 7 hours of time on stream. Even after the breakthrough, 70% of the HCl was captured by the adsorbent for a further extended period of time (7-12 hours).

There are numerous variations on the present invention which are possible in light of the teachings and supporting examples described herein. It is therefore understood that within the scope of the following claims, the invention may be practiced otherwise than as specifically described or exemplified herein.

What is claimed is:

1. A hydrodechlorination and hydrogen chloride recovery process, comprising:
a) contacting at least one hydrocarbon product with a hydrodechlorination catalyst in a presence of hydrogen under hydrodechlorination conditions to provide:
i) an off-gas comprising an HCl, and
ii) a dechlorinated product;
b) separating the dechlorinated product from the off-gas;
c) contacting the off-gas with an adsorbent under HCl adsorbing conditions such that the HCl is adsorbed by the adsorbent; and
d) after step c), recovering the HCl from the adsorbent.

2. The process according to claim 1, wherein the hydrodechlorination conditions comprise a reaction temperature in the range from about 300° F. (about 148.9 degree Celsius) to 750° F. (398.9 degree Celsius), a reaction pressure in the range from about 100 to 5000 psig, a liquid hourly space velocity (LHSV) feed rate in the range from about 0.1 to 50, and a hydrogen supply in the range from about 50 to 8000 standard cubic feet per barrel (SCFB) of the at least one hydrocarbon product.

3. The process according to claim 1, wherein the hydrodechlorination catalyst comprises an element selected from the group consisting of elements of Groups 6, 8, 9, 10, and 11, and their mixtures, present as metals, oxides or sulfides.

4. The process according to claim 1, wherein:
step b) comprises separating the dechlorinated product, as a liquid, at a temperature in the range from about 50° F. (about 10 degree Celsius) to 600° F. (315.6 degree Celsius), and
step d) comprises contacting the adsorbent with a recovery carrier gas, wherein the HCl is desorbed from the adsorbent.

5. The process according to claim 1, wherein the adsorbent is selected from the group consisting of a molecular sieve, a refractory oxide, an activated carbon, and combinations thereof.

6. The process according to claim 1, wherein the adsorbent comprises a molecular sieve selected from the group consisting of 3A, 4A, 5A, 13X, 13Y, USY, ZSM-5, ZSM-22, ZSM-23, ZSM-35, ZSM-48, MCM-22, MCM-35, MCM-58, SAPO-5, SAPO-11, SAPO-35, and VPI-5.

7. The process according to claim 1, wherein the at least one hydrocarbon product is selected from the group consisting of alkylate gasoline, diesel fuel, jet fuel, base oil, and mixtures thereof obtained by contacting at least one hydrocarbon reactant with an ionic liquid catalyst in a hydrocarbon conversion zone under hydrocarbon conversion conditions, wherein a first chloride content of the at least one hydrocarbon product is greater than 50 ppm, and a second chloride content of the dechlorinated product is less than about 10 ppm.

8. The process according to claim 1, wherein the off-gas additionally comprises C5+ hydrocarbons.

9. The process according to claim 1, wherein recovering the HCl from the adsorbent is done by desorption of the HCl at an ambient temperature.

10. The process according to claim 1, wherein recovering the HCl from the adsorbent is done by feeding a recovery carrier gas through a spent adsorbent bed.

11. The process according to claim 10, wherein the recovery carrier gas comprises dry N₂.
